(19) United States
(12) Patent Application Publication
(10) Pub. No.: US 2003/0104354 A1
(43) Pub. Date: Jun. 5, 2003

(54) Method and device for the rapid clinical diagnosis of human cytomegalovirus (hCMV) infection in biological samples

(75) Inventors: Vijay Sharma, Mumbai (IN); Venkata Ramana Kondiboyina, Mumbai (IN)

Correspondence Address: LACKENBACH SIEGEL, ONE CHASE ROAD, SCARSDALE, NY, 10583, US

(73) Assignee: Reliance Life Sciences Private Limited, Mumbai (IN)

(21) Appl. No.: 10/012,996

(22) Filed: Nov. 30, 2001

(51) Int. Cl. C12Q 1/70; C12Q 1/68; C12M 1/34

(52) U.S. Cl. 435/5; 435/287.2; 435/6

(57) ABSTRACT

The present invention is a method and kit for rapid clinical diagnosis of hCMV in which the amplimers are transcripts of a glycoprotein B neutralization-related epitope gene of hCMV. The amplicons are hybridized to a specific oligonucleotide probe, which allows the amplicons to be detected.

### BACKGROUND OF THE INVENTION

1. Field of Invention

The present invention relates to methods and devices for the diagnosis of infections. In particular, the present invention relates to methods and kits for detection of Human Cytomegalovirus Veremia (hCMV).

2. Description of the Prior Art

Human Cytomegalovirus (hCMV) is a significant pathogen in immuno-compromised patients. hCMV is a member of beta-herpesvirinae, a subfamily of the herpesvirinae, which includes herpes simplex virus type I and II, varicella-zoster virus (the virus that causes chicken pox), and Epstein-Barr virus (the virus that causes mononucleosis). These viruses share a characteristic ability to remain dormant within the body over a long period of time. The initial infection, which is usually without symptoms, is always followed by a prolonged infection, during which the virus resides in cells without causing detectable damage or biochemical illness. Although the factors controlling latency and re-activation are not completely understood, impairment of the body's immune system by medication or disease can consistently re-activate the virus.

hCMV is a DNA virus and is approximately 200 nm in diameter. hCMV infection is usually subclinical in the healthy host. However, hCMV infection in a immuno-compromised host or a developing fetus may result in localized and/or disseminated disease. Clinical manifestations of hCMV include pneumonia, retinitis, hepatitis, enteritis, and neurological disease. Despite improved treatment modalities, hCMV infection may result in significant morbidity and mortality in transplant patients, AIDS patients, and cancer patients, particularly those with leukemia or lymphoma. Patients are at risk from both primary hCMV infection and reactivation of latent infection.

Early and rapid diagnosis of hCMV infection is of great importance. Yet, conventional methods for detection of hCMV based on serological assays are inaccurate and/or slow. For example, shell vial cultures provide results within 1 to 2 days, but are not sensitive for detection of hCMV in blood specimens. Moreover, distinguishing asymptomatic viral shedding from a significant disease that requires treatment can be difficult. In particular, isolation of hCMV from saliva and urine is commonly uncorrelated with symptomatic disease, but isolation from blood specimens has been shown to correlate with symptomatic disease.

The following requirements need to be fulfilled for an optimal assay for hCMV diagnosis.

High sensitivity and specificity;

Rapid results; and

High reproducibility.

There have been developments in quantitative antigen detection methods, such as hCMV antigenemia assays and molecular amplification methods. The hCMV antigenemia assay is valuable in the diagnosis and monitoring of active hCMV infection in solid organ transplant patients and bone marrow transplant patients.

Kits for detection of hCMV are commercially available. One such kit is produced by Hoffmann-La Roche and sold under the trademark Amplicor. This kit makes use of amplification by Polymerase Chain Reaction (PCR) to create amplicons specific to a targeted hCMV gene followed by Enzyme Linked Immunosorbent Assay (ELISA) to detect the amplicons.

The drawback associated with prior art kits and methods lies in the sensitivity of the assay. Moreover, since amplimers are visualized with a naked eye, results can be highly variable. Furthermore, traditional systems for gel analysis cannot differentiate bands that have been co-amplified. Also, weak amplimers may not be detected, which results in a false-negative result. Thus a negative result does not exclude the possibility of hCMV infection.

In light of the foregoing, there is a need for a more sensitive, specific and rapid detection protocol for clinical samples. The inventors of the present invention have been successful in developing a kit and a method for detecting hCMV in a more sensitive, specific and rapid manner. The present invention obviates the problems associated with the conventional kits.

Several terms used in the invention are defined as follows.

The term “primer” refers to a synthetic oligonucleotide sequence synthesized for annealing to a specific nucleotide sequence of interest. The primer initiates DNA synthesis to occur using thermostable DNA dependent DNA polymerase. Selecting the proper primer is one of the most important steps in designing a PCR kit. The primer set must hybridize to the target sequence with little or no hybridization to other sequences that are also present in the sample.

The term “probe” refers to a synthetic oligonucleotide sequence which lies internal to the + strand of the amplified product resulting from a PCR reaction.

The term “hybridization” refers to annealing of nucleotide sequences to each other under optimal conditions. Typically, a nucleotide A binds to nucleotide T and nucleotide G binds to nucleotide C.

The term ‘biological samples’ refers to the samples selected from serum, urine, paraffin embedded tissue, plasma, whole blood, and combinations thereof. When selected, plasma and whole blood samples will preferably include an anticoagulant, such as EDTA and/or ACD.

### SUMMARY OF THE INVENTION

It is an object of the present invention to develop an improved hCMV diagnostic kit.

It is an object of the present invention to develop an improved method for rapid clinical diagnosis of hCMV infection.

It is still an object of the invention is to develop a method using specific primer sets and detection probe having higher sensitivity and specificity as compared to the conventional assays.

These and other objects of the present invention are substantially achieved by a method and kit for rapid clinical diagnosis of hCMV in which the amplimers are transcripts of a glycoprotein B neutralization-related epitope gene of hCMV. The amplicons are hybridized to a specific oligonucleotide probe, which allows the amplicons to be detected.

### DESCRIPTION OF THE INVENTION

Selecting the target DNA sequence of the Glycoprotein B neutralization-related epitope gene for hCMV depends upon identification of regions within the hCMV genome that show maximum sequence conservation among all the serotypes of hCMV.

The present invention has primers that are complementary to sequences flanking a segment of the Glycoprotein B neutralization-related epitope gene to be amplified. The primers of the present invention are selected for their ability to specifically recognize the Glycoprotein B neutralization-related epitope gene with a low mutation frequency.

The primers of the present invention are so designed to avoid hairpin loop structure formation. In addition, the selected primers have been subjected to a gene bank search to identify homologies and percent similarities to the target of interest. The results were as follows:

| SEQ. ID. NO. 1 specificities | SEQ. ID. NO. 2 specificities |
|---|---|
| 100% to HHV 5 | 100% to 1 HHV isolate |
| 100% to HCMV glycoprotein B | 100% to HCMV |
| 100% to human chromosome 13q | no homologue to human chromosome |
| | 13q |

A diagnostic kit for detection of hCMV in biological samples according to the present invention includes two components. The first component is the amplification component, which is used to amplify the target sequence. The second component of the kit is the detection component, which is used to detect the amplicons produced by the amplification component.

The amplification component amplifies the target sequence via PCR and, therefore, will include a pair of amplification oligonucleotide primers, thermostable DNA-dependent DNA polymerase; and deoxyribonucleoside triphosphates. The primers are labeled at their 5′ end. Labels are preferably selected from the group consisting of fluorescein, biotin, digoxigenin, and radioactive labels (e.g., ³²P). A more preferable label is fluorescein.

The first oligonucleotide primer for use in the amplification component has the following nucleic acid sequence (SEQ. ID. NO. 1):

5′-tgaggaatgtcagcttc-3′

SEQ. ID. NO. 1 contains seventeen (17) continuous bases selected from the glycoprotein B neutralization related epitope sense strand. The Nucleotide Sequence Position of SEQ. ID. 1 is 81347-81363. SEQ. ID. NO. 1 is preferably present in the amplification component in an amount of about 1 μl to about 10 μl.

The second oligonucleotide primer for use in the amplification component has the following nucleic acid sequence (SEQ. ID. NO. 2):

5′-tcatgaggtcgtccaga-3′

SEQ. ID. NO. 2 contains seventeen (17) continuous bases selected from the glycoprotein B neutralization related epitope antisense strand. The Nucleotide Sequence Position of SEQ. ID. NO. 2 is 81656-81672. SEQ. ID. NO. 2 is preferably present in the amplification component in an amount of about 1 μl to about 10 μl.

The annealing temperature of primer pair is generally calculated as 50° C. lower than the estimated melting temperature. The annealing temperature for primers that are less than 20 bases is calculated using the following formula: [4(G+C)+2(A+T)]−5° C. Ideally the annealing temperature of each primer should match and be within the 55° C. and 75° C. range. If the annealing temperature difference between the two primers is high, the lower annealing temperature can be increased adding to the length of that primer at either the 3′ end (this can also keep the size of the amplified locus constant) or the 5′ end. The annealing temperature for the primers of the present invention is about 58° C.

The thermostable DNA-dependent DNA polymerase may be any suitable thermostable DNA-dependent polymerase. Preferably, the thermostable DNA-dependent polymerase is derived from Thermus aquaticus (Taq) bacteria. The thermostable DNA-dependent polymerase is preferably present in the amplification component in an amount of about 1 unit to about 2.5 units.

The deoxyribonucleoside triphosphates (dNTPs) useful in the present invention include: dATP, dCTP, 5MedCTP, dGTP, dITP, TTP, and dUTP. Preferably, the dNTPs are selected from dATP, dCTP, dGTP, dTTP, and combinations thereof. Preferably, each dNTP is present in the amplification component in an amount of about 100 μmole to about 200 μmole.

The amplification component may contain any other suitable additional ingredient and/or component, such as an amplification buffer. For example, suitable 10× amplification buffers for use in the present invention include 100 mM Tris HCl (pH 8.3), 500 mM KCl, and MgCl₂.

A preferred amplification component includes:

(1) an amplification buffer having 10 mM Tris HCl (pH 8.3) and 500 mM KCl;

(2) about 100 to about 200 μM each of dATP, dCTP, dTTP and dGTP;

(3) sterile distilled water(nuclease free);

(4) about 1 unit to about 2.5 units of thermostable DNA-dependent DNA polymerase;

(5) about 10 to about 100 pM of the first oligonucleotide primer (SEQ. ID NO. 1);

(6) about 10 to about 100 pM of the second oligonucleotide primer (SEQ. ID NO. 2);

(7) about 1.5 to about 2.5 mM MgCl₂; and

(8) the template to be amplified.

Preferably, the volume of the amplification buffer is about 25 to about 50 μl and the volume of the extracted sample is about 25 to about 50 μl. The final volume of the amplification component is about 50 to about 100 μl.

The detection component preferably detects the amplified target sequence via ELISA and, therefore, will include a oligonucleotide probe immobilized upon a solid medium, a enzyme conjugate specific to the selected label on the amplicons, and a substrate that changes color in the presence of the enzyme on the conjugate.

The oligonucleotide probe is at least twenty continuous bases selected from the following a nucleic acid sequence (SEQ. ID. NO. 3):

5′-ttccagtaccctgaagtcggtattttc-3′

The Nucleotide Sequence Postion of SEQ. ID. NO. 3 is 81501-81528 and contains twenty-seven (27) continuous bases. SEQ. ID. NO. 3 is a region internal to the amplimers created using the primers of SEQ. ID. NO. 1 and SEQ. ID. NO. 2

SEQ. ID. NO. 3 is specifically designed for capturing the amplification product. The oligonucleotide probe is preferably immobilized on a solid medium, such as a microwell plate. For example, the oligonucleotide probe may be labeled at the 5′ end with biotin, which is substantially irreversibly bound to streptavidin coating the microwell plate.

The oligonucleotide probe is preferably present in the detection component in an amount of about 10 μl to about 100 μl.

Since the label on the amplicon is preferably fluorescein, the conjugate is preferably an anti-fluorescein/horse raddish peroxidase conjugate present in an amount of about 1 unit to about 4 units. However, any suitable conjugate may be used, depending only upon the label present on the amplicon.

The enzyme on the conjugate may be any enzyme, depending only upon the selected substrate. For example, the preferred enzyme for the present invention is horse raddish peroxidase (HRP). However, other enzymes, such as alkaline phosphatase, may be used.

The substrate changes color in the presence of the enzyme conjugate to visibly show the presence of the amplimer bound to the oligonucleotide probe. Thus, a change in the color of the detection solution positively indicates the presence of the amplimer and, by extension, the presence of hCMV in the original biological sample. The selection of the substrate is dependent upon the selected enzyme on the conjugate. A preferred enzyme and substrate combination for use in the present invention is peroxidase and a mixture of hydrogen peroxide (H₂O₂) and 3,3′,5,5′-Tetra methyl benzidine Dihydrochloride (TMB) present in an amount of about 100 microliters, which is oxidized by H₂O₂ in the presence of peroxidase and, thus, the detection solution changes from colorless to blue. Other suitable enzyme and substrate combinations are as follows:

Alkaline Phosphatase and 5-Bromo-4Chloro-3Indolyl Phosphate (BCIP)

Alkaline Phosphatase and Fast Red RC

Alkaline Phosphatase and Naphthol AS-TR Phosphate

Alkaline Phosphatase and Nitro Blue Tetrazolium (NBT)

Alkaline Phosphatase and p-Nitrophenyl Phosphate (pNPP)

Peroxidase and 3-Amino-9-Ethylcarbazole (AEC)

Peroxidase and 5-Aminosalicyclic acid (5AS)

Peroxidase and 2,2′-Azino-bis(3-Ethylbenzthiazoline-6-Sulfonic acid)

Peroxidase and 4-Chloro-1-Naphthol (4CIN)

Peroxidase and 3-3′Diaminobenzidine Tetrahydro-chloride (DAB)

Peroxidase and o-Dianisidine

Peroxidase and o-Phenylenediamine Freebase (OPD)

A method for rapid clinical diagnosis hCMV according to the present invention uses the first primer (SEQ. ID. NO. 1) and the second primer (SEQ. ID. NO. 2) in an amplification step, and the oligonucleotide probe (SEQ. ID. NO. 3) in a detection step.

In accordance with a second aspect of this invention, a method according to the present invention includes the steps of sample extraction, amplification (preferably by PCR), and detection by enzyme immunoassay (preferably ELISA).

hCMV nucleic acid is extracted from a biological sample, preferably using chaotropic agents, such as urea, diethylamine, guanidium hydrochloride, potassium iodide, sodium dodecyl sulphate (SDS), Formamide, and combinations therefo. Any suitable and/or known technique for extraction of nucleic acid may be used.

The extracted specimen is then added to the amplification component. As discussed above, the amplification component contains the primers of the present invention (SEQ. ID. NOs. 1 and 2) having a label at their 5′ ends, deoxyribonucleoside triphosphates, and a thermostable DNA-dependent DNA polymerase.

Amplification is accomplished by repeated cycles of: DNA denaturation, primer annealing, and extension of the primed DNA sequence by the DNA polymerase in the presence of added purine and pyrimidine bases. In general, each cycle will double the amount of the target DNA sequence. The amplification cycle is repeated until a detectable amount of the DNA sequence has been created. Further details of the PCR method are provided in U.S. Pat. Nos. 4,683,195; 4,683,202; 4,965,188; and 5,075,216, which are incorporated herein by reference in their entirety.

The amplimers are preferably detected using ELISA. Denatured and labeled amplimers are added to a microwell containing the immobilized oligonucleotide probe of the present invention (SEQ. ID. NO. 3) and a hybridization solution, thereby immobilizing the amplimers by hybridization with the oligonucleotide probe. An anti-label conjugate with a selected enzyme is added to the microwell after excess unbound amplimers are washed away. Finally, a substrate is added to the microwell, which changes color in the presence of the enzyme on the anti-label conjugate. Thus, the detection solution changes color if any amplimers are present in the microwell, which denotes a positive result for the presence of hCMV in the original biological sample.

This method is rapid, automatable (ELISA type solid phase formats do not require gel electrophoresis) and applicable to large scale screening programs.

The following example illustrates the process according to the invention without limitation.

### EXAMPLE 1

An experiment was conducted to demonstrate the method of present invention. The initial sample taken was plasma EDTA that was pre-determined as positive for the presence of hCMV mediated antibodies (IgG, IgM) and Hepatitis B Surface Antigen (HbSAg). The sample (about 0.2 ml) was extracted using the a DNA isolation system made by Qiagen Inc., Venlo, The Netherlands, and sold under the trademark QIAamp®.

The amplification reaction was set up using 25 μL of the above extracted nucleic acid and 25 μL of 10× amplification buffer including 100 mM Tris HCl (pH 8.3), 500 mM KCl, and MgCl₂ was added to make a final concentration of about 1.5 mM to about 2.5 mM. The primers (SEQ. ID. NOs. 1 and 2) were labeled with fluorescein and provided in a concentration of about 10 pM. Each dNTP (dATP, dCTP, dGTP, and dTTP) was provided in a concentration of about 100 μM to about 200 μM. Taq polymerase was provided in an amount of about 1 unit to about 2.5 units.

This reaction mixture was heated to 94° C. for 10 min followed by 30 amplification cycles. Each amplification cycle included heating the reaction mixture to 94° C. for about 30 sec, cooling to 62° C. for 45 sec, and heating to 72° C. for 30 sec. Following the 30 amplification cycles, a final incubation was performed for 10 min at 72° C. The resulting amplimers were denatured using a solution of 0.4 M NaOH.

The detection of the fluorescein labeled amplimers was done in the following manner:

50-100 microliters of dilution buffer was pipetted into a microwell plate coated with streptavidin, to which was added about 10 microliters of the oligonucleotide probe (SEQ. ID. NO. 3) labeled with biotin. The solution was incubated at 37° C. for 30-60 min. Thereafter, the microwell plate was washed with PBS-T wash buffer, and 100 microliters of a hybridization buffer was added and incubated for 15-30 min at 37° C. The hybridization buffer included sodium phosphate, sodium thiocyanade, and Denhardts solution, which included polyvinylpyrollidine (PVP), Ficoll, and Bovine Serum Albumin (BSA)

25 microliters of denatured amplification product was added to the microwell plate and incubated for about 30 to about 60 min at 42° C. Following incubation, the microwell plate was washed 5 times with PBS-T wash buffer. About 100 microliters of diluted anti-fluorescein-HRP conjugate was added to the washed plate and incubated for 30-45 min at 37° C. Following incubation, the microwell plate was washed 5 times with PBS. About 100 microliters of TMB containing hydrogen peroxide was added to the microwell plate and incubated substantially without light at room temperature for 15 to 30 minutes.

A color change from colorless to blue was observed after incubating for about 15 min. 50 microliters of a stop solution containing 0.1 NH₂SO₄ was added and the change in color from blue to yellow was noticed and the plate was read at 403 nm in a colorimetric plate reader.

In view of the foregoing descriptions and example, it will become apparent to those of ordinary skill in the art that equivalent modifications thereof may be made without departing from the spirit and scope of this invention. Various features are set forth in the following claims.

We claim:

1. A device for amplifying hCMV nucleic acid comprising:
a first amplification primer having the sequence:
5′-tgaggaatgtcagcttc-3′
and a second amplification primer having the sequence:
5′-tcatgaggtcgtccaga-3′.

2. The device according to claim 1, wherein the first amplification primer comprises at least seventeen continuous bases selected from the glycoprotein B neutralization related epitope sense strand.

3. The device according to claim 1, wherein the first amplification primer is present in an amount of about 10 pmole to about 100 pmole.

4. The device according to claim 1, wherein the second amplification primer comprises at least seventeen continuous bases selected from the glycoprotein B neutralization related epitope anti-sense strand.

5. The device according to claim 1, wherein the second amplification primer is present in an amount of about 10 pmole to about 100 pmole

6. The device according to claim 1, further comprising thermostable DNA-dependent DNA polymerase.

7. The device according to claim 6, wherein the DNA polymerase is Taq polymerase present in an amount of about 1 unit to about 2.5 units.

8. The device according to claim 1, further comprising a deoxyribonucleoside triphosphate.

9. The device according to claim 8, wherein said deoxyribonucleoside triphosphate is selected from the group consisting of: dATP, dCTP, 5MedCTP, dGTP, dITP, TTP, dUTP, and combinations thereof.

10. The device according to claim 8, wherein said deoxyribonucleoside triphosphate is present in an amount of about 100 to about 200 μmoles.

11. The device according to claim 1, wherein the first and second amplification primers have a label at their respective 5′ ends.

12. The device according to claim 11, wherein the label is fluorescein.

13. A device for detecting an hCMV nucleic acid comprising an oligonucleotide probe having the sequence:
5′-ttccagtaccctgaagtcggtattttc-3′.

14. The device according to claim 13, wherein the sequence of the oligonucleotide probe is internal to an amplimer resulting from an amplification using primers SEQ.ID.NO. 1 and SEQ.ID.NO. 2.

15. The device according to claim 13, wherein the oligonucleotide probe has a label at its 5′ end.

16. The device according to claim 15, wherein the label is biotin

17. The device according to claim 13, wherein the oligonucleotide probe is immobilized on a solid medium.

18. The device according to claim 13, wherein the oligonucleotide probe is present in an amount of about 50 pmole to about 100 pmole.

19. The device according to claim 13, further comprising a conjugate adapted to bind with a selected label present on the hCMV nucleic acid.

20. The device according to claim 19, wherein the selected label is fluorescein and the conjugate is an anti-fluorescein/horse raddish peroxidase conjugate present in an amount of about 1 unit to about 4 units.

21. The device according to claim 19, further comprising a substrate adapted to change color in the presence of an enzyme on the conjugate.

22. The device according to claim 21, wherein the substrate comprises hydrogen peroxide and 3,3′,5,5′-tetramethylbenzidine dihydrochloride.

23. The device according to claim 22, wherein the substrate is present in an amount of about 100 microliters.

24. A method for detecting hCMV nucleic acid in a biological sample comprising the steps of:
extracting hCMV nucleic acid a biological sample;
amplifying the hCMV nucleic acid using a first primer having the sequence
5′-tgaggaatgtcagcttc-3′
and a second primer having the sequence
5′-tcatgaggtcgtccaga-3′;
and detecting the hCMV nucleic acid using an oligonucleotide probe having the sequence:
5′-ttccagtaccctgaagtcggtattttc-3′.

25. The method according to claim 24, wherein the biological sample is selected from the group consisting of:serum, plasma, whole blood, urine, paraffin embedded tissue, and combinations thereof.

26. The method according to claim 24, wherein the first and second amplification primers have a label at their respective 5′ ends.

27. The method according to claim 26, wherein the label is fluorescein.

28. The method according to claim 24, wherein amplifying the hCMV nucleic acid comprises the steps of:
denaturing the hCMV nucleic acid to produce denatured hCMV nucleic acid;
annealing the first and second amplification primers to the denatured hCMV nucleic acid to produce primed hCMV nucleic acid; and
extending the primed hCMV nucleic acid using a thermostable DNA-dependent DNA polymerase in the presence of a deoxyribonucleoside triphosphate.

29. The method according to claim 28, wherein the DNA polymerase is Taq polymerase present in an amount of about 1 unit to about 2.5 units.

30. The method according to claim 28, wherein the deoxyribonucleoside triphosphate is selected from the group consisting of: dATP, dCTP, 5MedCTP, dGTP, dITP, TTP, dUTP, and combinations thereof, and wherein the deoxyribonucleoside triphosphate is present in an amount of about 100 μmoles to about 200 μmoles.

31. The method according to claim 24, wherein detecting the hCMV nucleic acid comprises the steps of:
binding the hCMV nucleic acid with the oligonuclotide probe attached to a solid medium to form immobilized hCMV nucleic acid;
binding the immobilized hCMV nucleic acid with a conjugate;
adding a substrate that is adapted to change color in the presence of an enzyme on the conjugate,
whereby a change of the color of the substrate indicates the presence of hCMV nucleic acid.

32. The method according to claim 31, wherein the hCMV nucleic acid is labeled with flourescein, and wherein the conjugate is an anti-flourescein/horse raddish peroxidase conjugate in an amount of about 1 unit to about 4 units.

33. The method according to claim 31, wherein the substrate comprises hydrogen peroxide and 3,3′,5,5′-tetramethylbenzidine dihydrochloride.

34. The method according to claim 31, wherein the substrate is present in an amount of about 100 microliters.

35. The method according to claim 31, further comprising the step of reading a change of the color of the substrate with a colorimetric plate reader.
